(19) United States
(12) Patent Application Publication
(10) Pub. No.: US 2021/0280308 A1
(43) Pub. Date: Sep. 9, 2021

(54) APPLYING BIOMETRICS TO THE DEVELOPMENT OF DIGITAL CONTENT

(71) Applicant: Nervanix, LLC, Naples, FL (US)

(72) Inventor: Adam Leonard Hall, Fort Myers, FL (US)

(21) Appl. No.: 17/193,931

(22) Filed: Mar. 5, 2021

Related U.S. Application Data
(60) Provisional application No. 62/985,700, filed on Mar. 5, 2020.

(51) Int. Cl. G16H 40/63

(52) U.S. Cl. CPC G16H 40/63

(57) ABSTRACT

A method includes providing digital content to a content designer through a device and receiving an input from the content designer through the device indicating a desire to tag an event in the digital content. A user interface is then provided to receive a predicted biometric response from the content designer, the predicted biometric response comprising a biometric response that is expected to be observed in end users when the end users experience the event. An identifier for the event and the predicted biometric response is then stored so that it is associated with the digital content.

### CROSS-REFERENCE TO RELATED APPLICATION

The present application is based on and claims the benefit of U.S. provisional patent application Ser. No. 62/985,700, filed Mar. 5, 2020, the content of which is hereby incorporated by reference in its entirety.

### BACKGROUND

Modern instructional materials include digital content consisting of text, video, and interactive exercises. Students use electronic devices to experience the digital content and for some materials, students control their progression through the materials by selecting what they wish to view next.

Designers of such instructional materials begin with a set of objectives that they want to achieve through the materials. For example, these objectives may be the retention of particular information by the student or may be a desired emotional response to a set of circumstances depicted in the materials.

The discussion above is merely provided for general background information and is not intended to be used as an aid in determining the scope of the claimed subject matter. The claimed subject matter is not limited to implementations that solve any or all disadvantages noted in the background.

### SUMMARY

A method includes providing digital content to a content designer through a device and receiving an input from the content designer through the device indicating a desire to tag an event in the digital content. A user interface is then provided to receive a predicted biometric response from the content designer, the predicted biometric response comprising a biometric response that is expected to be observed in an intended audience when the intended audience experiences the event. An identifier for the event and the predicted biometric response is then stored so that it is associated with the digital content.

In accordance with a further embodiment, a method includes, for each of a plurality of digital content, receiving a plurality of biometric responses to events that occur during interactions with the digital content. The plurality of biometric responses for the plurality of digital content are then used to construct a model of biometric responses to events. Information about new digital content and a tagged event in the new digital content is received and the model is used to generate a model biometric response to the event.

In accordance with a still further embodiment, a method includes providing digital content with tagged events to an end user and determining when a tagged event is triggered in the digital content. A type of biometric to be measured for the triggered event is retrieved and the biometric of the end user is measured. A variance between the measured biometric and a predicted biometric response for the tagged event is determined.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is flow diagram of a method of tagging digital content with events.

FIG. 2 is a block diagram of a system used in the method of FIG. 1.

FIG. 3 is a user interface for adding events to digital content.

FIG. 4 is a user interface for adding attributes that define a click event.

FIG. 5 is a user interface for adding a hypothesis to an event.

FIG. 6 is the user interface of FIG. 5 showing the addition of a biometric to an event.

FIG. 7 shows a change in the user interface after a click event has been added.

FIG. 8 is a user interface for adding attributes that define a change in URL event.

FIG. 9 shows a change in the user interface after a URL event has been added.

FIG. 10 is a user interface for adding attributes that define a video event.

FIG. 11 is a user interface for adding attributes that define an image event.

FIG. 12 is an alternative user interface for tagging events in digital content.

FIG. 13 is an alternative user interface for setting attributes for an event.

FIG. 14 is an alternative user interface for setting attributes for an event.

FIG. 15 is a block diagram of a system for collecting actual biometric response to digital content and comparing the actual biometric responses to predicted biometric responses.

FIG. 16 is a method performed by the system of FIG. 15.

FIG. 17 is a report user interface showing actual and predicted biometrics for an event.

FIG. 18 is flow diagram of a method of generating and using artificial intelligence models to evaluate digital content.

FIG. 19 is a block diagram of a computing system used to implement various embodiments.

### DETAILED DESCRIPTION

Current systems do not provide a means for digital content designers to evaluate the impact that their digital material has on end users. In particular, current systems do not provide a way to measure the engagement or emotional response of end users to particular events that occur in the materials. This makes it difficult for the content designer to understand what aspects of their materials need improvement.

The present embodiments provide systems and methods that allow a content designer to tag particular events in the digital material so that biometric responses to those events can be recorded. In addition, the systems and methods allow the content designer to enter a prediction of the biometric response they expect. This helps the content designer to honestly assess whether a particular portion of their materials is attaining the attention levels, emotional responses, memory activation or overall engagement that the content designer had hoped for when creating the materials.

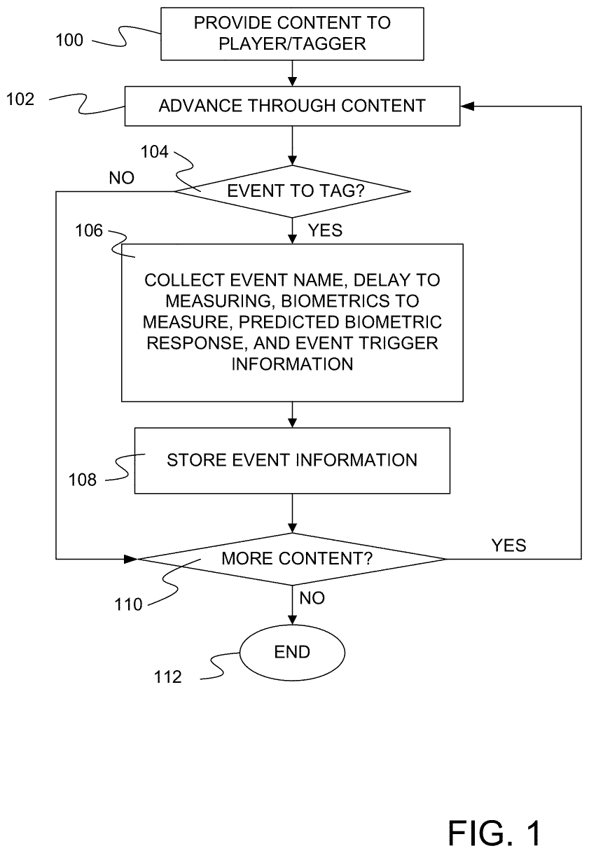

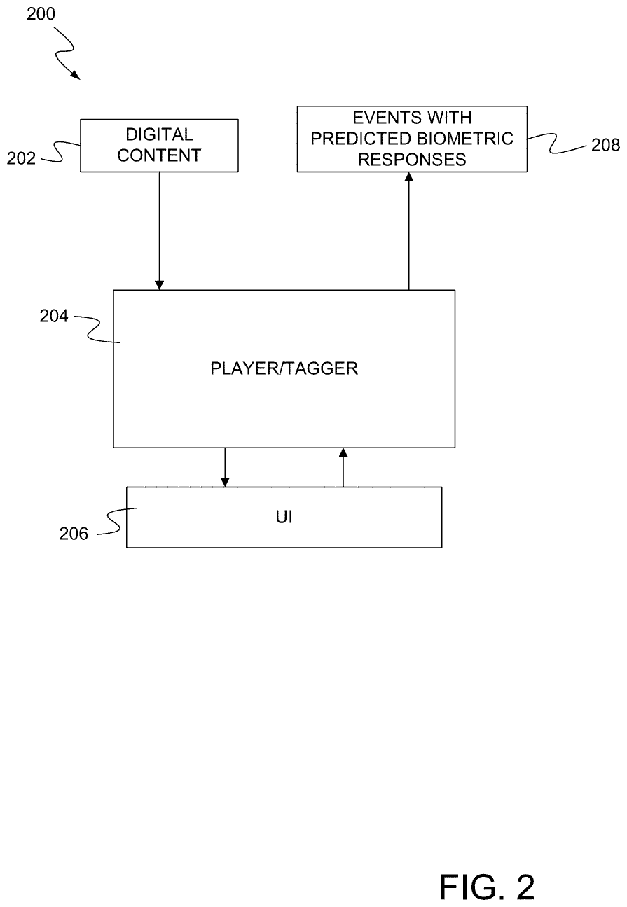

FIG. 1 provides a flow diagram of a method of tagging events in accordance with one embodiment. FIG. 2 provides a block diagram of a system 200 for tagging events in accordance with the embodiment of FIG. 1. In step 100 of FIG. 1, digital content 202 is provided to a player/tagger application 204. Player/tagger application 204 allows the content designer to advance through the digital content using a user interface 206 at step 102. Thus, the content designer is able to click on controls in the content, select URL links shown in the content, play videos provided in the content, and listen to and view the content. In accordance with one embodiment, a click on a control, a change in URL, the playing of a video, and the displaying of an image can each be used to set an event. At step 104, the content designer indicates that they wish to tag an event through user interface 206. In accordance with one embodiment, the content designer will want to tag an event when the content designer expects a particular biometric response from end users when the end users are exposed to the event. When the content designer indicates that they wish to tag an event, the user interface is updated with a screen that allows the content designer to enter information about the event and the predicted biometric response. In particular, at step 106, player/tagger 204 provides controls on user interface 206 to collect the following: the event type (automatically set by player/tagger 204 in most embodiments), an event identifier that will allow the event to be detected (also automatically set by player/tagger 204 in most embodiments), a delay between the event and when biometric measurements should begin, the biometrics to be measured, a name for the event, a predicted biometric response to the event, and a hypothesis for the predicted biometric response.

Different embodiments use different event identifiers. For example, the event identifier can be an identifier for a displayed control in combination with an event such as an identifier of a button in combination with a “click” on the button. In such systems, access to the underlying code or script of the digital content is required. The event identifier can also be a video play control or an image in a video playback window. The event identifier can also be a Uniform Resource Locator (URL) that is providing the digital content. In still further embodiments, the event identifier can be an image or part of an image displayed on the screen. In such embodiments, a copy of an image of the screen or part of the screen is stored for later comparison to images produced on the screen when the end user is progressing through the content. In one particular embodiment, innocuous markers such as QRcodes are provided at different inflection points of the digital content. Since the QRcodes can be readily tracked and interpreted by software, such as the Python Imaging Library (PIL), the QRcode present during the event can be used as the event identifier.

The biometrics to be measured and the predicted biometric response are important to capture before the digital content is evaluated. This helps to reduce the amount of biometric data that needs to be returned to the content designer and helps the content designer to perform an honest assessment of the performance of the digital content. By recording the content designer's prediction of the biometric response, the system provides expectations that the actual biometric responses can be compared against. When the actual biometric responses do not match the predicted biometric response, the content designer must accept that the digital content did not perform as expected making it more likely that the content designer will revise the digital content to achieve the objectives for the digital content.

The predicted biometric response can be a specific value provided by a biometric sensor or a change in a value provided by the biometric sensor. The biometric response can also be a combination of values or changes in values provided by a plurality of biometric sensors at the same time. Some changes in biometric sensor signals are complex waveforms that must be analyzed to determine the biometric response. For example, the biometric sensor signals can be analyzed to determine that the end user's biometric response involves a change in Attention, Emotion, Memory or Engagement. To assist the content designer, a list of possible biometric responses, including neuro-metric responses, is provided in user interface 206 so that the content designer does not have to memorize the various biometric responses that are possible.

The hypothesis for the predicted biometric response describes what is significant in terms of the digital content presentation and how that presentation should be expected to affect the end user's body and brain. The hypotheses define the context and expectations for the biometric behavior consistent with the content designer's intentions regarding how the digital content should perform—biometrically.

This is the chief function the content designer needs in a system; a way to develop a narrative of behavior around metrics that are well understood. While the actual engagement process with digital content (such as “learning”) cannot be directly observed with current neural science, many of the cues that the content designer wants to manipulate can be. So the narrative that develops the hypothesis needs to be disciplined in hard, established meaning. However, the language associated with the neural cues is hardly standardized. While well-established concepts like attention and emotion may be understood intuitively, newer concepts, like the correlation of neural activity among subjects, are still evolving. Thus, Cross-Brain Correlation, Inter Subject Correlation, and Engagement are all terms in the literature that represent different flavors of this nascent metric.

Another consideration when attempting to build a standardized tool for integrating biometrics into digital content evaluation is the content designer's familiarity with neuro-terminology. It is assumed that a content designer has a deep understanding of the issues involved in digital content development, but not necessarily a similar familiarity with brain science concepts, or other biometric concepts. This, coupled with the fact that a “biometric coach”, such as a neuroscientist, cannot always be on hand, supports the argument that a system providing a selection of standardized language corresponding to the biometric cues can provide a significant advantage. To accomplish this standardization, the system uses current descriptive language in the biometric fields (such as neuroscience) often used to categorize metric graphs. In addition, excerpts from papers that describe biometric behavior from previous studies are used as prompts to keep the content designers on track as they characterize events for current and future studies. Additionally, the system provides access to tutorials about the metrics. Having this language built into system 200 is instrumental in keeping the content designer in synch with the biometric sciences. Also, using standard language allows meaningful comparisons between the hypothesized result and the description of the actual metric observed. Another benefit of having standardized language is the ability to use more advanced analytics methods. When the language and observations of renowned biometric scientists can be organized in a library for formulating conjectures and reporting behavior, machine learning classification algorithms can be used to attach the metric graphs to the corresponding language. When a new graph is presented for analysis, the appropriate language can be retrieved to compare to the hypothesis. Thus, the various embodiments incorporate cutting edge research and make it available as an established standard.

FIG. 2 provides an example of user interface 206 displayed by player/tagger 204. User interface 206 includes a digital content window 300 and an events window 302 that has a menu 304. Events window 302 will provide a list of events defined for the digital content shown in digital content window 300. Digital content window 300 includes video links 306, 308, 310 and 312 and video playback window 314.

Menu 304 includes a set of icons 320, 322, 324, and 326, each representing a separate type of event that can be set for the digital content. In accordance with some embodiments, not all the icons shown in menu 304 will always be provided. In particular, if the digital content does not provide information needed to implement a particular event type, the event type's icon will not be shown. For example, if the digital content does not provide information that will allow player/tagger 204 to detect a control “click”, icon 326, which is for a “click” type event, will not be displayed.

Icon 320 is for an image event in which the event is triggered based on the current image shown in digital content window 300. Icon 322 is for a video event in which the event is either triggered by the start of a video in playback window 314 or by an image shown in playback window 314. Icon 324 is for a URL event in which the event is triggered by a change to a particular URL that the digital content is provided from. Icon 326 is for a click event in which a control in digital content window 300 is “clicked” or otherwise selected.

The content designer interacts with the digital content in digital content window 300 in the same manner that an end user will interact with the digital content at step 102. When the content designer wants to add an event to the digital content at step 104, the content designer selects one of icons 320, 322, 324 and 326.

For example, when the content designer wants to add a click event, the content designer first selects click icon 326 and then selects (clicks) the control in digital content window 300 that is to trigger the event. After the control is selected, a screen 400 is displayed to the content designer so that the content designer can enter information about the event. Screen 400 includes a label input area 402, an event name input area 404, a delay input area 406 that defines a delay between when the event occurs and when the biometric data is to be recorded for the event, a frequency input 408 that defines how often clicking on the control should trigger recording of the biometric data, and an “ADD HYPOTHESIS” button 410.

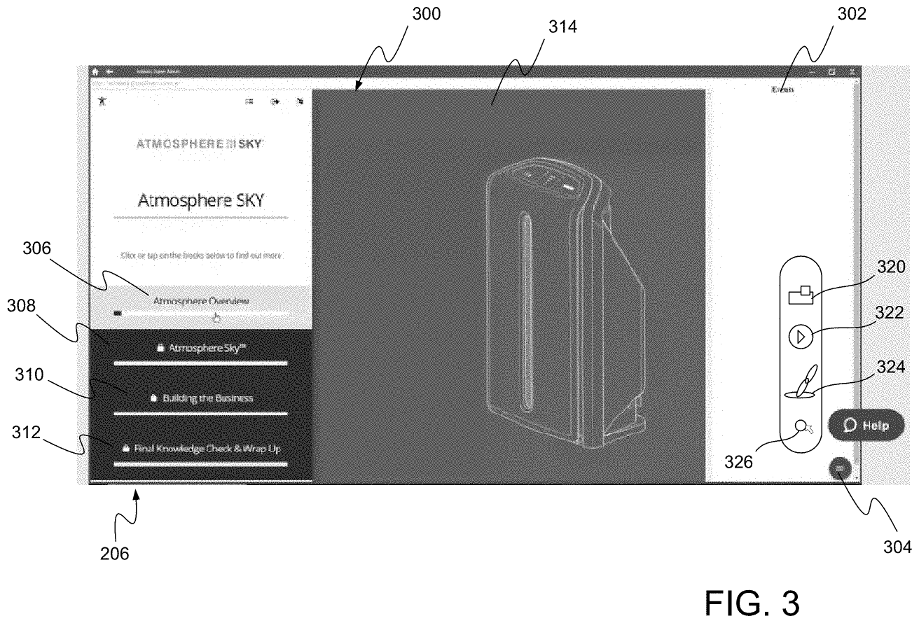

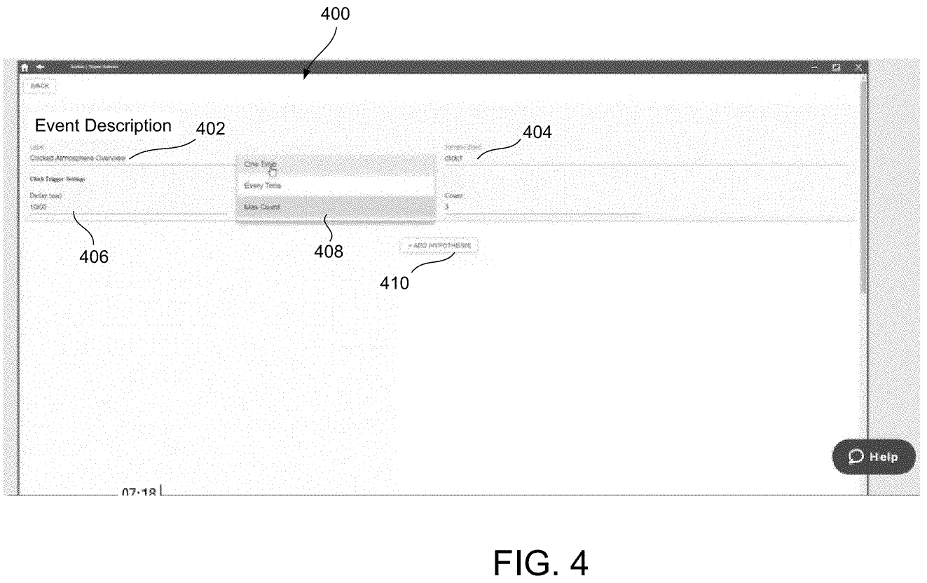

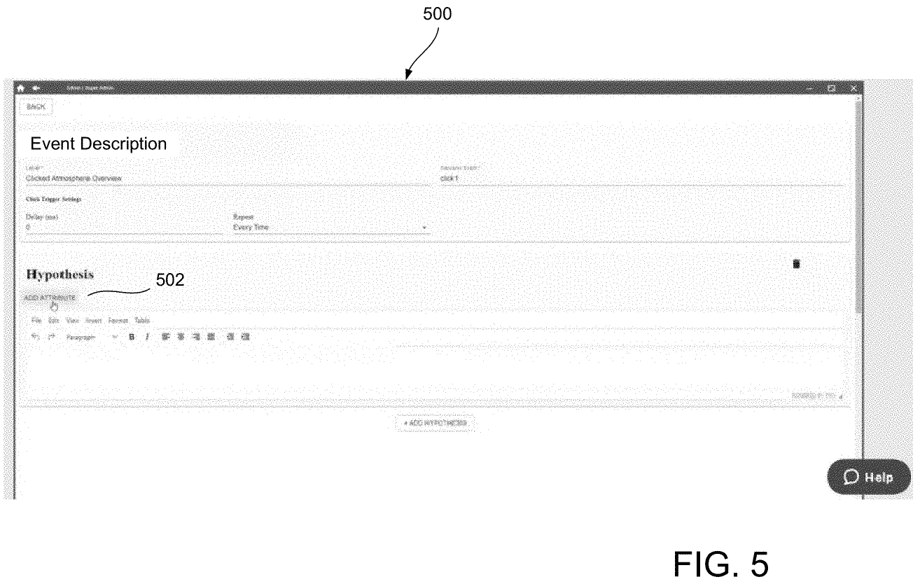

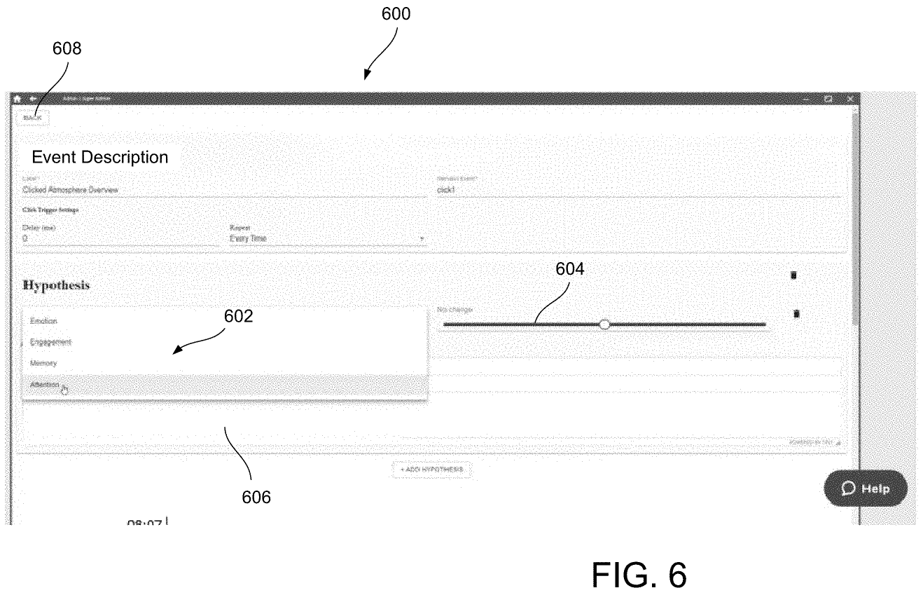

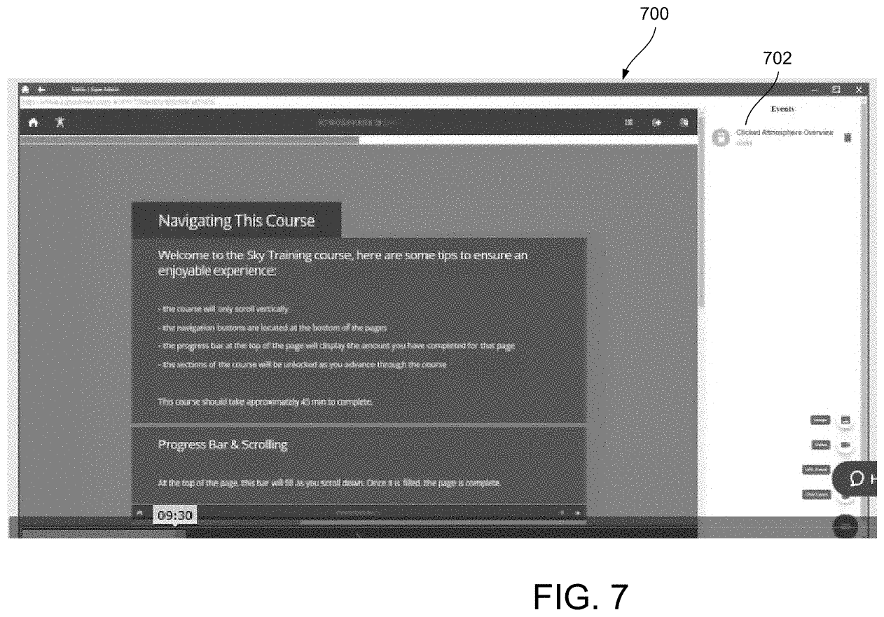

When “ADD HYPOTHESIS” button 410 is selected, screen 500 of FIG. 5 is displayed on user interface 206. Screen 500 includes an “ADD ATTRIBUTE” button 502. When “ADD ATTRIBUTE” button 500 is selected, screen 500 changes to screen 600 of FIG. 6, where the content designer is able to select one or more biometrics from a displayed list of biometrics 602, set an expected change for the selected biometric using control 604, and type a description of the expected change and a hypothesis describing the reason the biometric response is expected in a text box 606. In accordance with some embodiments, possible terms to describe the metric behavior are provided for the content designer from a library. FIG. 7 shows an example screen 700 of user interface 206 showing the addition of a click event 702 that was defined in FIGS. 3-6. When a back button 608 is selected, the identity of the control selected by the content designer and the event information are stored in events storage 208.

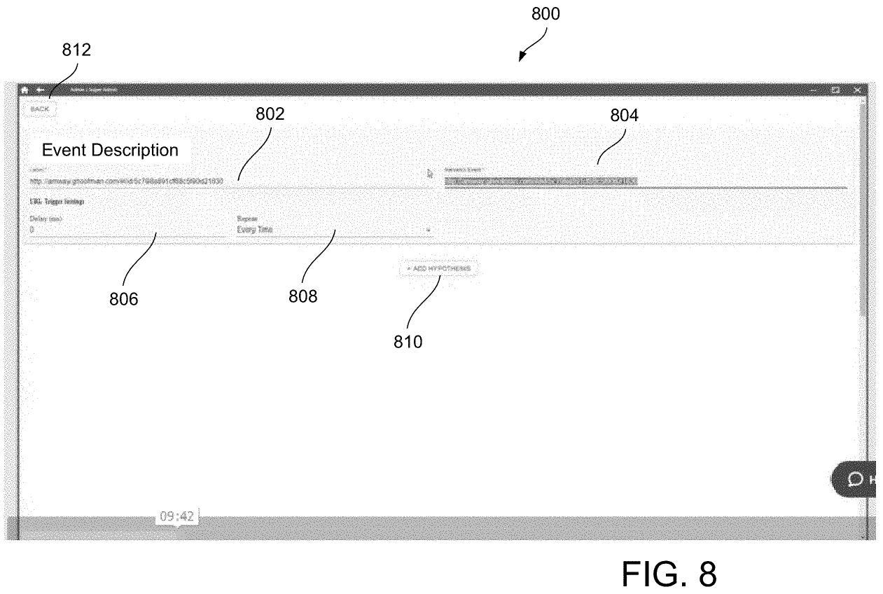

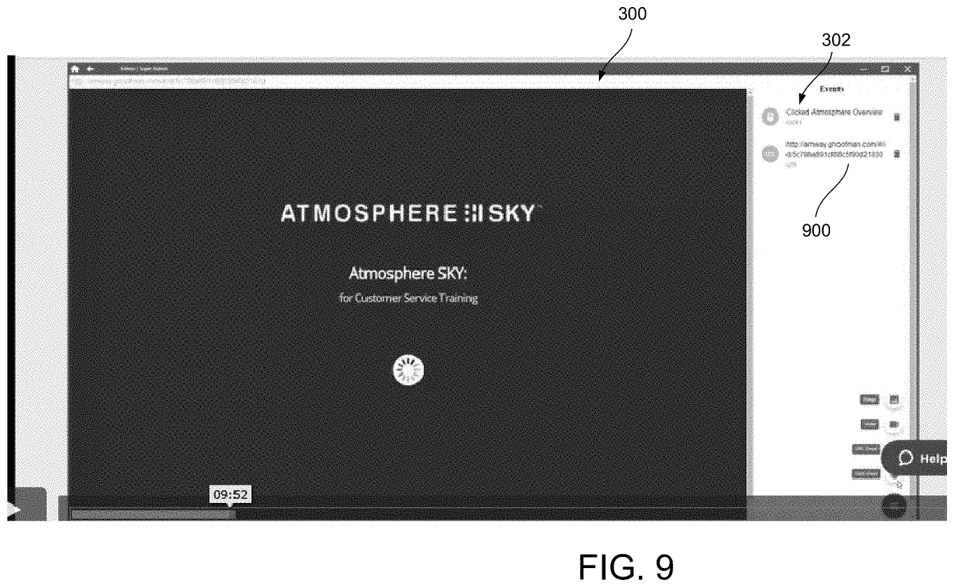

The content designer can also add a URL event using icon 324 of menu 304. When icon 324 is selected, of the current URL providing content to digital content window 300 is captured and stored for the event. A screen, such as screen 800 of FIG. 8, is then displayed on user interface 206 for the content designer to enter details about the event including a label 802, an event name 804, a delay 806 between when the event occurs and when the biometric data is to be recorded for the event and how often 808 the change in the URL should trigger recording of the biometric data. FIG. 8 also includes an “ADD HYPOTHESIS” button 810 that when selected allows the content designer to select a biometric, the expected change in the biometric when the event occurs and a description of the event. When a back button 812 is selected, the URL and the event information are stored in events storage 208. FIG. 9 shows an example user interface showing the addition of a URL event 900 in events window 302.

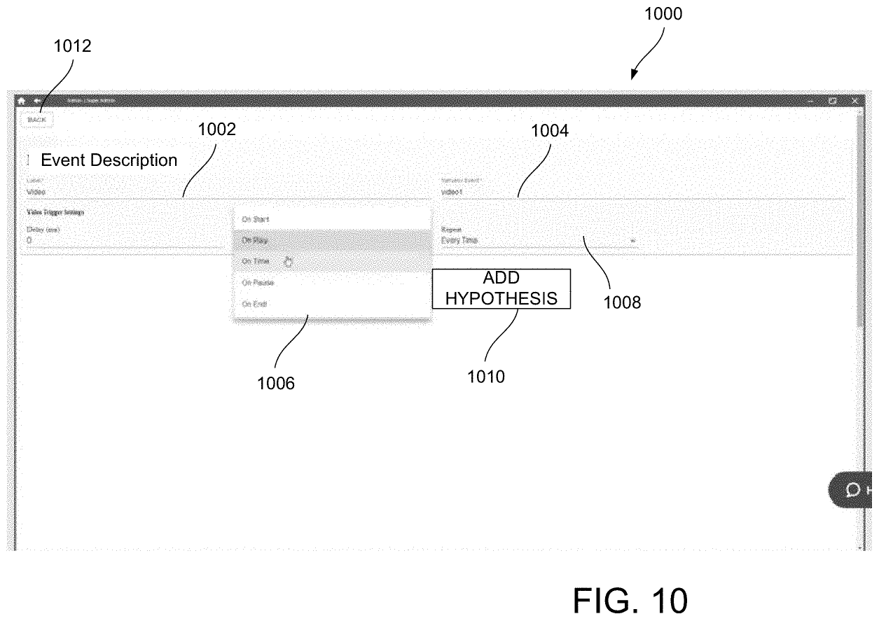

The content designer can also add a video event by selecting video icon 322 in menu 304. In accordance with one embodiment, after selecting icon 322, the content designer selects a video play button for the video that is to trigger the event. Information about the video play button is stored, such as a button identifier or a URL of the video, and then a screen is displayed to allow the content designer to enter information about the video event. For example, screen 1000 of FIG. 10 is displayed. Screen 1000 includes a label input area 1002, an event name input area 1004, a timing input area 1006 that defines when the biometric data is to be recorded for the event, a frequency input 1008 that defines how often clicking on the control should trigger recording of the biometric data, and an “ADD HYPOTHESIS” button 1010. Timing input 1006 allows the content designer to designate a time for recording the biometric data that is relative to the video playback such as when the play button is clicked, when the video actually begins to play, when the video is paused, or when the video ends. The content designer can also designate a time delay after when the play button is pressed for the biometric data to be recorded. When “ADD HYPOTHESIS” button 1010 is selected, additional elements are added to the screen to allow the content designer to select one or more biometrics to be recorded, the expected change in the biometrics when the event occurs and a description of reasoning for the predicted biometric response. When a back button 1012 is selected, the identity of the video selected by the content designer and the event information are stored in events storage 208.

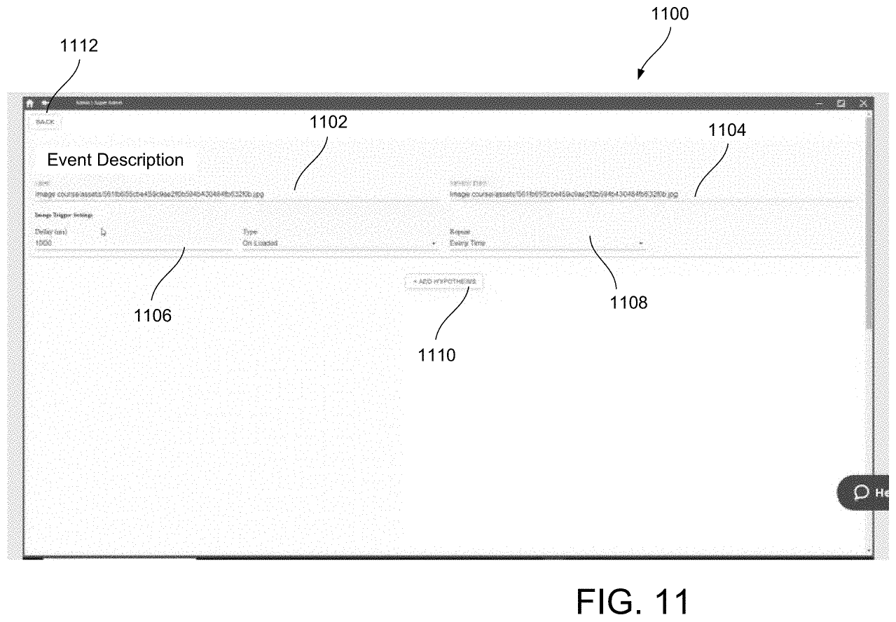

The content designer can also add an image event by selecting image icon 320. When image icon 320 is selected, the current image displayed in digital content window 300 is captured and screen 1100 of FIG. 11 is displayed for the content designer to enter details about the event including a label 1102, an event name 1104, an indication of when the biometric data is to be recorded for the event 1106 relative to when the digital content window matches the stored image and how often the event should trigger recording of the biometric data 1108. FIG. 11 also includes an “ADD HYPOTHESIS” button 1110 that when selected allows the content designer to select a biometric, the expected change in the biometric when the event occurs and a description of the reasoning behind the predicted biometric response. When a back button 1112 is selected, the image of digital content window 300 and the event information are stored in events storage 208.

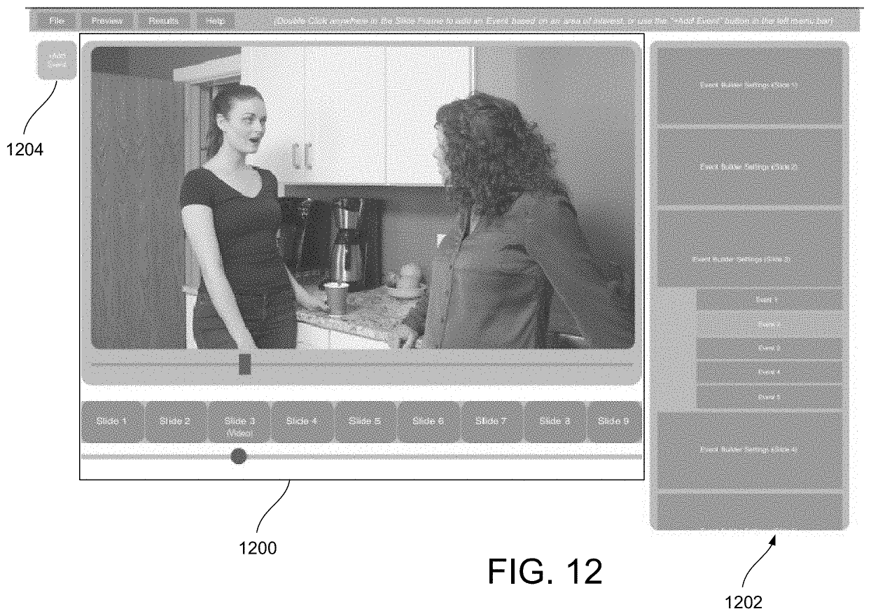

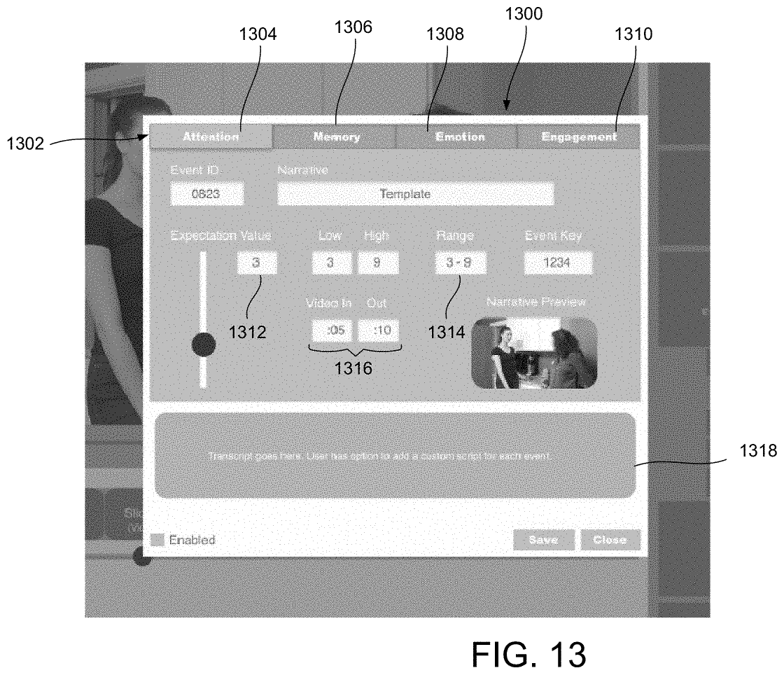

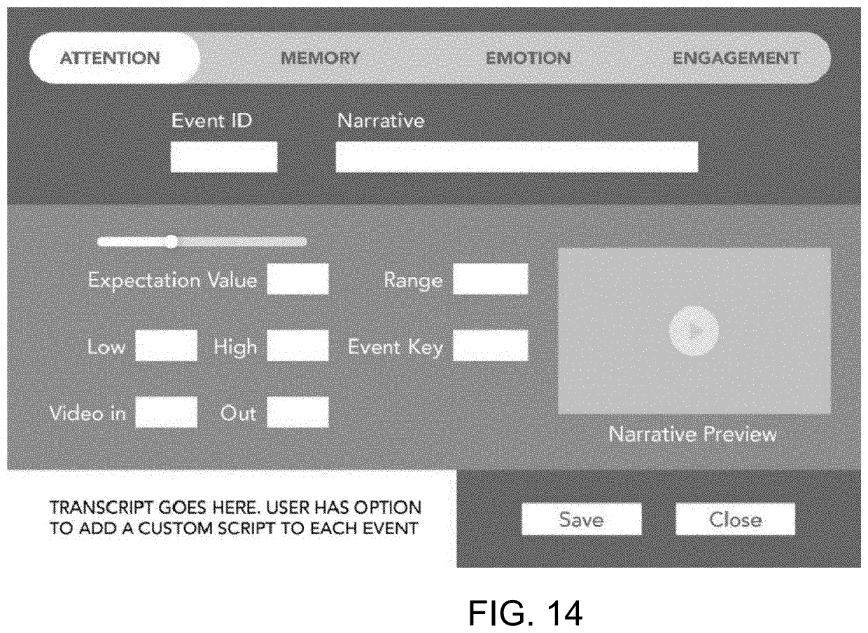

FIG. 12 provides an alternative user interface for selecting events. In FIG. 14, a window 1200 shows the current digital contents. In the right-hand side is an area 1202 where previously designated events are listed. An add event control 1204 is used by the content designer to add a new event. When add event control 1204 is selected to define a new event, screen 1300 of FIG. 13 is provided to receive the attributes of the event. Screen 1300 includes a row of tabs 1302 at the top that allow the content designer to select one or more biometrics 1304, 1306, 1308 and 1310 for the event. For each selected biometric, the content designer can enter an expected value 1312 for the biometric during the event, a range 1314 of possible values for the biometric, a time period 1316 during which the biometric data is to be recorded for the event, one or more event identifiers and labels, and a written description 1318 of the expected biometric response during the event. FIG. 14 provides an alternative version of FIG. 13.

Returning to FIGS. 1 and 2, at step 108, the event information 208 is stored so that it is associated with digital content 202. After step 108, or if an event is not to be tagged, player/tagger 204 determines if there is more content at step 110. If there is more content, player/tagger 204 continues to allow the content designer to advance through the content by returning to step 102. When there is no more content at step 110, the process of FIG. 1 ends at step 112.

Once digital content has been tagged with events, the digital content can be evaluated by having end users (representative samples of intended audiences) progress through the digital content while collecting biometric data from those end users. These data sets are acquired in lab settings where representative samples of human subjects experience the digital content and are measured with varying degrees of biometric sensors. Depending on the combination of applied biometric sensors, appropriate/relevant algorithms are applied to the biometric data sets to establish biometrics, such as neuro-metric mind states (eg. Attention, Memory, Emotion, Engagement, Fear, Loathing, Risk Aversion, etc.)

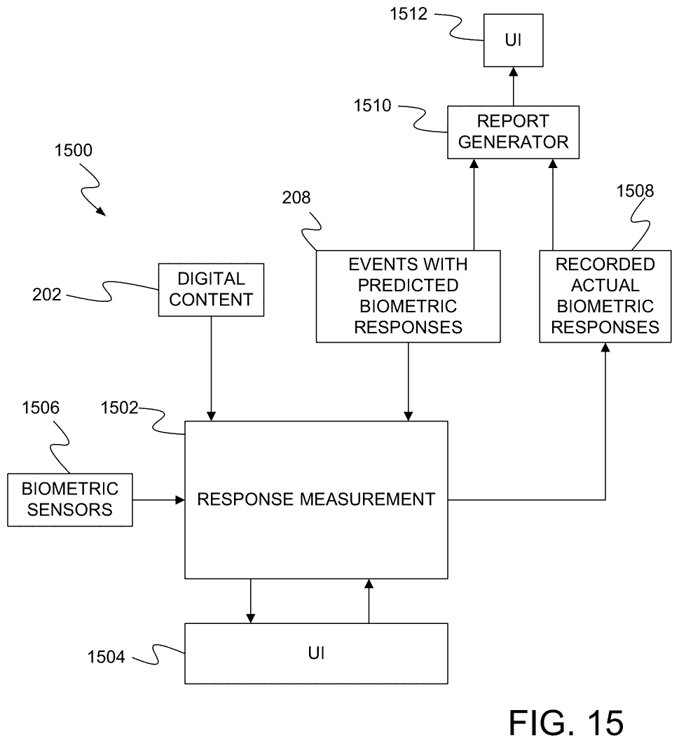

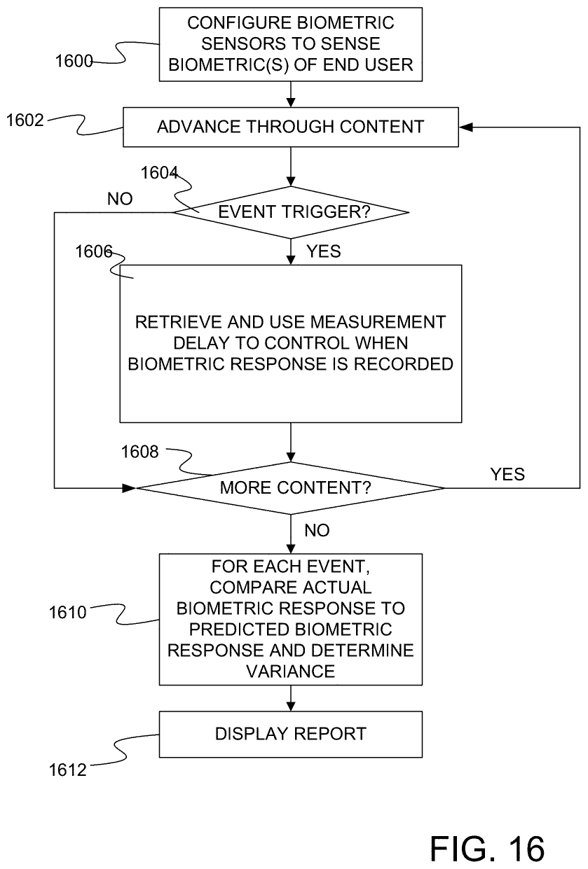

FIG. 15 provides a block diagram of a system 1500 for recording biometric data while end users experience tagged content and evaluating the content based on differences between actual biometric responses and predicted biometric responses. FIG. 16 provides a flow diagram of a method implemented on system 1500.

In step 1600, one or more biometric sensors 1506 are configured to capture biometrics of end users. Examples of possible biometrics sensed by sensors 1506 include:

Electroencephalography (EEG), which is an electrophysiological monitoring method to record electrical activity of the brain. It is typically noninvasive, with electrodes placed along the scalp, EEG measures voltage fluctuations resulting from ionic current within the neurons of the brain, Clinically, EEG refers to the recording of the brain's spontaneous electrical activity over a period of time, as recorded from multiple electrodes placed on the scalp. Diagnostic applications generally focus either on event-related potentials or on the spectral content of EEG. The former investigates potential fluctuations time locked to an event, such as ‘stimulus onset’ or ‘button press’. The latter analyses the type of neural oscillations (popularly called “brain waves”) that can be observed in EEG signals in the frequency domain.
GSR (Galvanic Skin Response) aka: EDA (Electrodermal activity), which is the property of the human body that causes continuous variation in the electrical characteristics of the skin. The traditional theory of EDA holds that skin resistance varies with the state of sweat glands in the skin. Sweating is controlled by the sympathetic nervous system, and skin conductance is an indication of psychological or physiological arousal. If the sympathetic branch of the autonomic nervous system is highly aroused, then sweat gland activity also increases, which in turn increases skin conductance, In this way, skin conductance can be a measure of emotional and sympathetic responses.
Eye Gaze, which is the point where a person is looking or the motion of an eye relative to the head. An eye tracker is a device for measuring eye positions and eye movement. There are a number of methods for measuring eye movement. The most popular variant uses video images from which the eye position is extracted. Other methods use search coils or are based on the electrooculogram.
ECG (Electrocardiography), which is a measure of the electrical activity of the heart using electrodes placed on the skin. These electrodes detect the small electrical changes that are a consequence of cardiac muscle depolarization followed by repolarization during each cardiac cycle (heartbeat). In a conventional 12-lead ECG, ten electrodes are placed on the patient's limbs and on the surface of the chest. The overall magnitude of the heart's electrical potential is then measured from twelve different angles (“leads”) and is recorded over a period of time (usually ten seconds). In this way, the overall magnitude and direction of the heart's electrical depolarization is captured at each moment throughout the cardiac cycle,
Electromyography (EMG), which is a measure of the electrical activity produced by skeletal muscles. EMG is performed using an instrument called an electromyograph, which detects the electric potential generated by muscle cells when these cells are electrically or neurologically activated.

In one example of a lab session, end users wear EEG senor arrays while they consume digital content. In accordance with one embodiment, the EEG data are collected using a 32-channel EEG system (Brain Products GmbH, Gilching, Germany) at a rate of 500 samples per second and a measurement range of ±341.6 mV with a gain factor of 12. The device uses an internal battery (capacity, 1,000 mAh; approximately 4 hours of recording at 500 mA) that is charged before the experiment to reduce line noise. The device itself applies a common-mode rejection for artifacts greater than 80 dB, and a low-pass filter inside the amplifier of 131 Hz. The data are recorded as 24-bit samples with a resolution of approximately 40.7 nano-Volt per bit. Beyond the 32-channels EEG data, the study collects three additional 3-axis acceleration data, which were used for artifact rejection based on notable head movements. The data are transmitted from the acquisition device wirelessly to a nearby server as well as recorded locally on a 32-Gb micro-SD memory card for backup in case of wireless failure. The data are exported as three files. The EEG samples are recorded in European Data Format (EDF) file. The two additional files included: a header file with basic information about the recording (recording start time, date, impedance, sampling rate, etc.) and a marker file that included the annotation of events that occurred during the acquisition (i.e., “acquisition started”). The dry electrodes are coated with silver chloride (chemical formula: AgCl) using an actiCAP Xpress Twist. An amplifier was attached to the acquisition cap on a dedicated back pocket to minimize external noise. The amplifier weighs approximately 30 grams including the cable connecting the LiveAmp amplifier to the electrodes. The acquisition is done locally on the server running a Window 10 Operating System and a proprietary software, BrainVision Recorder 1.21.

Subjects are fitted with an EEG electrode cap with a circumference of either 54 or 58 centimeters depending on head size and comfort with the cap's tightness prior to the data acquisition. The electrodes used are either flat electrodes touching the surface of the skin—primarily for frontal sites, and pin-electrodes with flexible tip that were made to penetrate the hair. The electrodes depths vary between 12 and 14 mm and are replaced during the calibration to assure subject comfort. The facilitators of the study select the electrodes such that the signal is clean within each site. If neither 12 nor 14-mm electrodes yielded a clear signal upon visual inspection, the facilitators apply a washable conductive gel with a syringe at the electrode tip to improve the quality of the acquisition data. Each electrode connection is verified to be functioning properly (i.e. capturing electrical activity from the scalp) before starting the recording.

The electrode locations are distributed across the entire scalp according to the actiCap 32Ch Standard-2 (green holders) montage, which correspond to the 10-20 International system.

Data are pre-processed using Matlab's EEGLab extension. None of the pre-processing is done manually to allow for perfect reproducibility of the results. The random number generators implemented in the artifact removals functions of EEGLab are reset to a common seed to remove any randomness.

To process the data, a low-pass filter at 0.75 Hz and a high-pass filter at 50 Hz (function: pop_eegfiltnew) are applied. Filtering is done using Hamming windows Finite Impulse Response (FIR) filter. The clean_artifacts function is subsequently used to remove artifacts using the following criteria (maximum tolerated flatline duration of 5 seconds; minimum channel correlation of 0.85; line noise standard deviation of 4; an aggressive strict burst criterion of 5). Portions of the data that have more than 25% of the samples flagged as noise are removed entirely. Channels that have more than 10% of the data flagged as artifacts are marked as unusable.

Finally, the signal in each channel is referenced to an average reference across all the remaining electrodes. After the data has been pre-processed, it is used to calculate the various neuro-metrics selected for each event.

In order to generate the neural metrics a number of steps are taken based on the specific metric. For the neuro-metric “emotion”, Frontal Alpha Asymmetry is implemented, where the activity at Alpha band (generated using standard Fourier transformation of the raw signal to the frequency domain) is measured from frontal electrodes (typically channels F3/F4 and F7/F8 on the left/right hemispheres). The power across left-right electrodes is compared within a selected time frame (i.e., 10-second video recording). Asymmetry (i.e., greater values from the left/right channels) indicates emotional characteristics that are typically labeled “Approach” (for positive emotion) or “Avoidance” (for negative emotion). The data can be generated in real-time using a small lag in the data collection proportional to the window used for the analyses.

For the neuro-metric “memory”, gamma/theta activation proportion difference between the left/right hemisphere (with temporal electrodes T7/T8 and T9/T10 typically used for the analyses) is used as a measure of memory activation.

For the neuro-metric “attention”, the percent increase/decrease of alpha power from baseline activation (with baseline established in “resting state” recording prior to the video viewing; typically about 30 seconds of recordings with eyes-open followed by 30-seconds with eyes-closed without external stimulus) is measured. The attention is measured from occipital electrodes (O1/O2/Oz) primarily.

Finally, the neuro-metric “engagement” is generated by estimation of Cross-subject Correlation (CBC) as established in U.S. Pat. No. 9,280,784. CBC is established by correlating the alpha power activity across numerous electrodes (based on the montage) within dyads of brains. That is, pairs of EEG recording matrices are correlated channel-by-channel, and averaged across the entire scalp acquisition to yield a single number (per timepoints) reflective of the alignment and synchrony between brains at a given moment. Over time the fluctuations of CBC reflect the similarity between brains of multiple content viewers (i.e., viewers of content using the embodiments). Averaging the CBC across all pairs (n-choose-2 data points) yields a single metric of engagement by the group. This measure of all-brain alignment is proven to predict interest, memorability, and performance of the content viewed.

In accordance with some embodiments, additional neuro-metric measures of “risk-tolerance”, “anxiety”, “extroversion”, “neuroticism”, and “under high cognitive load” are determined.

Once the biometric sensors have been configured, response measurement application 1502 begins to provide digital content 202 to the end user through user interface 1504. While providing digital content 202, response measurement application 1502 monitors digital content 202 to determine if any events listed in events storage 208 occur in digital content 202 or in the end user's interaction with digital content 202. In one embodiment, this monitoring involves examining each control selection (click) to see if it triggers an event, examining each video play selection to see if it triggers an event, examining each change in URL to see if it triggers an event, and comparing each new image produced by the digital content to images stored in events storage 208 to see if the image triggers an event.

When an event is triggered at step 1604, response measurement application 1502 retrieves the information stored for the event in events storage 208 and uses the information to control what biometrics to record and when to start recording those biometrics at step 1606. In accordance with one embodiment, the biometrics are recorded for 20 seconds. A 20 second epoch scale provides a granularity that allows for specific significant biometric-observation and limits the range of stimuli in the digital content object to manageable content designer issues. The recorded biometrics are stored as recorded actual biometric responses 1508, which are associated with digital content 202 and the event that triggered the recording. In addition, the biometric responses are used to determine neuro-metric measures such as measures of “emotion”, “memory”, “attention” and engagement as described above.

At step 1608, response measurement application 1502 determines if there is more digital content, if there is more digital content, the process of FIG. 16 returns to step 1604 to await the next event trigger. When the content finishes at step 1608, the recorded actual biometric responses 1508 and corresponding neuro-metric measures, if any, can be compared to the predicted biometric response and predicted neuro-metric measures, if any, in events storage 208 by a report generator 1510 at step 1610. In some embodiments, this comparison produces a variance between the predicted biometric responses and the actual biometric responses. At step 1612, report generator 1510 generates a report for one or move events on a user interface 1512, which is viewed by the content designer to determine the performance of the content relative to the predicted performance. In accordance with one embodiment, the report is generated by aggregating all the actual biometric responses and neuro-metric measures across all the tested subjects and running analytics against the aggregate responses/measures to provide variances between the predicted biometric responses/neuro-metric measures and the aggregate biometric responses/neuro-metric measures for each event.

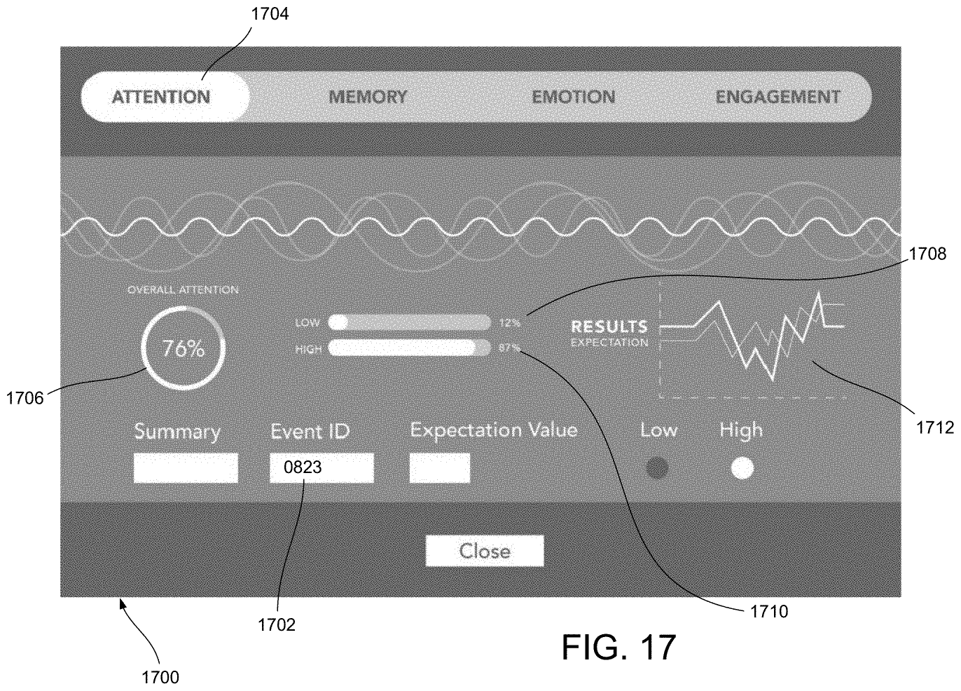

FIG. 17 provides an example of a report 1700 for an event. In FIG. 17, an event ID 1702 identifies the event and a biometric selection 1704 indicates the biometric that was measured. An indicator 1706 provides an average for the biometric across all subjects. An indicator 1708 provides the percentage of subjects that had actual biometric responses below the predicted biometric response and while an indicator 1710 provides a percentage of subjects that had actual biometric responses above the predicted biometric response. A graph 1712 indicates the variance between the average actual biometric response and the predicted biometric response over a time period.

In the discussion above, events are tagged by the content designer. In further embodiments, additional possible events are identified from the actual biometric responses of the subjects. In particular, the recorded biometric data is analyzed to find times when a particular biometric was significantly different from its average value. The digital content is then examined to determine what was occurring in the digital content during these times. This information is then provided to the content designer to suggest to the content designer that an additional event be added to the evaluation.

In the embodiments above, digital content is evaluated by having subjects experience the content while their biometric responses are recorded. This is an expensive and time-consuming process. In accordance with a further embodiment, an Artificial Intelligence model is constructed to take the place of the subjects thereby reducing the time and effort needed to evaluate the effectiveness of digital content.

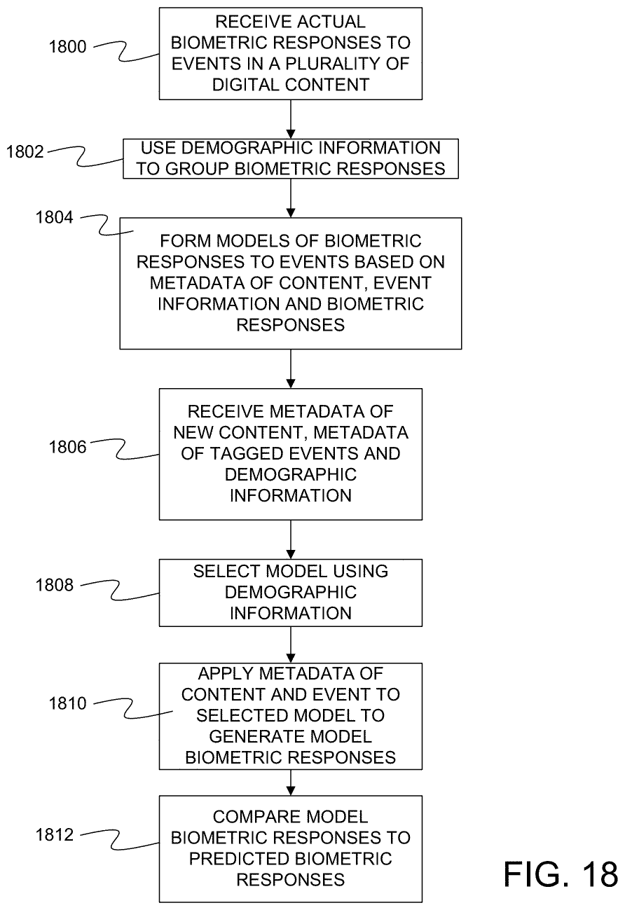

FIG. 18 provides a flow diagram of a method of producing and using models to evaluate the effectiveness of digital content. In step 1800, actual biometric responses to tagged events in a plurality of different digital content are received. Metadata for each event, such as the type of event, metadata describing the type of digital content, and metadata for the biometric response, such as the type of biometric, are provided with the actual biometric responses. In addition, demographic information about each end user is received with the end user's biometric responses. This demographic information can include multiple different demographic dimensions including, for example, age, gender, race, and education.

At step 1802, the biometric responses are grouped based on individual demographic dimensions or groups of demographic dimensions. At step 1804, the grouped biometric responses, the metadata for the content and the metadata for the events are used to generate a model of biometric responses for the corresponding demographic dimension(s) of the group. For example, a neural-net can be trained based on the provided information such that given metadata about new digital content and metadata about a tagged event in the new digital content, the neural-net will provide a model biometric response that is the model's estimate of the average biometric response end users would have had to the event.

At step 1806, metadata for new digital content and metadata for events tagged in the new content is received along with demographic information of likely end users. At step 1808, the demographic information is used to select one of the trained models and at step 1810, the metadata for the content and the metadata for the tagged events are applied to the model to generate a model biometric response for each tagged event. At step 1812, the model biometric response is compared to the predicted biometric response and variances between the responses are identified.

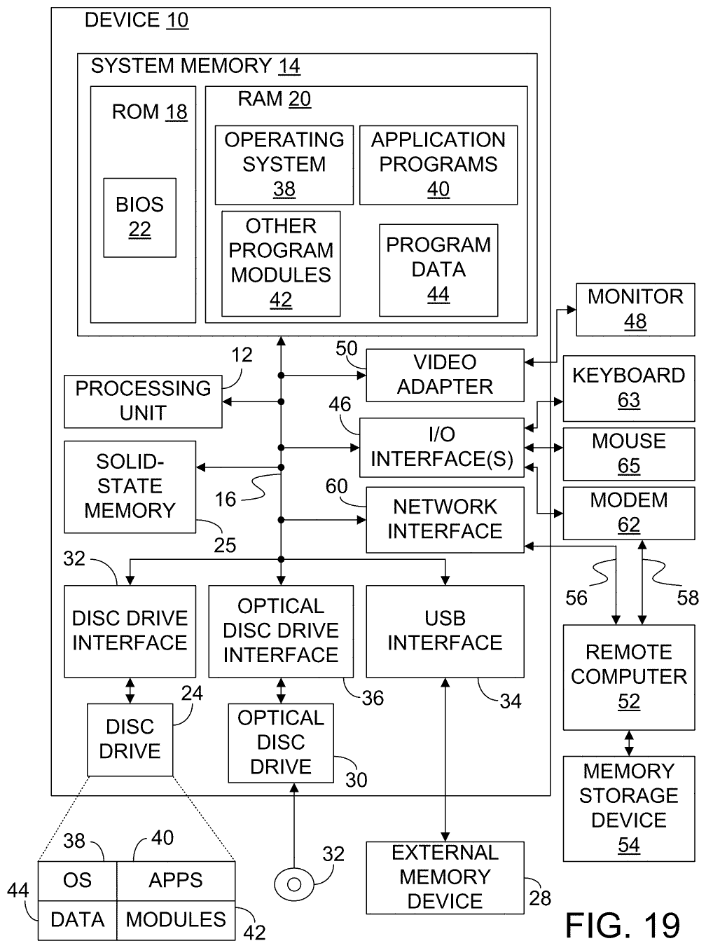

FIG. 19 provides an example of a computing device 10 that can be used to implement the systems discussed above. Computing device 10 includes a processing unit 12, a system memory 14 and a system bus 16 that couples the system memory 14 to the processing unit 12. System memory 14 includes read only memory (ROM) 18 and random-access memory (RAM) 20. A basic input/output system 22 (BIOS), containing the basic routines that help to transfer information between elements within the computing device 10, is stored in ROM 18. Computer-executable instructions that are to be executed by processing unit 12 may be stored in random access memory 20 before being executed.

Computing device 10 further includes an optional hard disc drive 24, an optional external memory device 28, and an optional optical disc drive 30. External memory device 28 can include an external disc drive or solid-state memory that may be attached to computing device 10 through an interface such as Universal Serial Bus interface 34, which is connected to system bus 16. Optical disc drive 30 can illustratively be utilized for reading data from (or writing data to) optical media, such as a CD-ROM disc 32. Hard disc drive 24 and optical disc drive 30 are connected to the system bus 16 by a hard disc drive interface 32 and an optical disc drive interface 36, respectively. The drives and external memory devices and their associated computer-readable media provide nonvolatile storage media for the computing device 10 on which computer-executable instructions and computer-readable data structures may be stored. Other types of media that are readable by a computer may also be used in the exemplary operation environment.

A number of program modules may be stored in the drives and RAM 20, including an operating system 38, one or more application programs 40, other program modules 42 and program data 44. In particular, application programs 40 can include programs for implementing any one of the applications discussed above. Program data 44 may include any data used by the systems and methods discussed above.

Processing unit 12, also referred to as a processor, executes programs in system memory 14 and solid-state memory 25 to perform the methods described above.

Input devices including a keyboard 63 and a mouse 65 are optionally connected to system bus 16 through an Input/Output interface 46 that is coupled to system bus 16. Monitor or display 48 is connected to the system bus 16 through a video adapter 50 and provides graphical images to users. Other peripheral output devices (e.g., speakers or printers) could also be included but have not been illustrated. In accordance with some embodiments, monitor 48 comprises a touch screen that both displays input and provides locations on the screen where the content designer is contacting the screen.

The computing device 10 may operate in a network environment utilizing connections to one or more remote computers, such as a remote computer 52. The remote computer 52 may be a server, a router, a peer device, or other common network node. Remote computer 52 may include many or all of the features and elements described in relation to computing device 10, although only a memory storage device 54 has been illustrated in FIG. 19. The network connections depicted in FIG. 19 include a local area network (LAN) 56 and a wide area network (WAN) 58. Such network environments are commonplace in the art.

The computing device 10 is connected to the LAN 56 through a network interface 60. The computing device 10 is also connected to WAN 58 and includes a modem 62 for establishing communications over the WAN 58. The modem 62, which may be internal or external, is connected to the system bus 16 via the I/O interface 46.

In a networked environment, program modules depicted relative to the computing device 10, or portions thereof, may be stored in the remote memory storage device 54. For example, application programs may be stored utilizing memory storage device 54. In addition, data associated with an application program may illustratively be stored within memory storage device 54. It will be appreciated that the network connections shown in FIG. 19 are exemplary and other means for establishing a communications link between the computers, such as a wireless interface communications link, may be used.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

1. A method comprising:
providing digital content to a content designer through a device;
receiving an input from the content designer through the device indicating a desire to tag an event in the digital content;
providing a user interface to receive a predicted biometric response from the content designer, the predicted biometric response comprising a biometric response that is expected to be observed in end users when the end users experience the event; and
storing an identifier for the event and the predicted biometric response so as to be associated with the digital content.

2. The method of claim 1 wherein providing the user interface comprises providing a list of selectable biometric types and input controls for designating how a selected biometric type is predicted to change during the biometric response.

3. The method of claim 1 wherein providing the user interface further comprises providing a control for selecting when a biometric response is to be measured.

4. The method of claim 1 wherein the control for selecting when the biometric response is to be measured comprises a control for selecting a time relative to when the event takes place.

5. The method of claim 1 wherein storing an identifier for the event comprises storing an image that appears on a display during the event.

6. The method of claim 1 further comprising:
providing the digital content to an end user;
detecting an actual biometric response from the end user for an event; and
storing the actual biometric response.

7. The method of claim 6 further comprising comparing the actual biometric response to the predicted biometric response and providing an indication of a variance between the actual biometric response obtained from the end user and the predicted biometric response.

8. The method of claim 7 further comprising:
using actual biometric responses for a plurality of end users to construct a model of biometric responses for content;
receiving new content; and
using the model of biometric responses for content to determine model biometric responses for the new content.

9. The method of claim 8 further comprising comparing a model biometric response to a predicted biometric response for the new content and indicating a variance between the model biometric response and the predicted biometric response for the new content.

10. The method of claim 6 further comprising:
detecting an actual biometric response for an untagged event;
providing an indication of the actual biometric response and a control for tagging the event; and
receiving an input through the control indicating a desire to tag the event.

11. A method comprising:
for each of a plurality of digital content, receiving a plurality of biometric responses to events that occur during interactions with the digital content;
using the plurality of biometric responses for the plurality of digital content to construct a model of biometric responses to events;
receiving information about new digital content and a tagged event in the new digital content; and
using the model to generate a model biometric response to the event in the new digital content.

12. The method of claim 11 further comprising:
receiving a predicted biometric response for the tagged event; and
providing an indication of a difference between the model biometric response and the predicted biometric response.

13. The method of claim 11 wherein receiving a biometric response in the plurality of biometric responses further comprises receiving demographic information of an end user that generated the biometric response.

14. The method of claim 13 wherein constructing the model comprises constructing the model for end users who have similar demographic information.

15. The method of claim 14 further comprising receiving demographic information and using the demographic information to select the model to use to generate the model biometric response.

16. A method comprising:
providing digital content with tagged events to an end user;
determining when a tagged event is triggered in the digital content;
retrieving a biometric to be measured for the triggered event;
measuring the biometric of the end user; and
determining a variance between the measured biometric and a predicted biometric response for the tagged event.

17. The method of claim 16 wherein determining when a tagged event is triggered comprises comparing an image on the digital content to a stored image.

18. The method of claim 16 further comprising retrieving a delay period between when the tagged event is triggered and when the biometric is to be measured wherein measuring the biometric of the end user comprises waiting for the delay period after the tagged event is triggered before measuring the biometric of the end user.

19. The method of claim 16 wherein measuring the biometric comprises measuring the biometric over a period of time.

20. The method of claim 16 further comprising using the measured biometric to train a model.
